(19) United States
(12) Patent Application Publication
(10) Pub. No.: US 2024/0189197 A1
(43) Pub. Date: Jun. 13, 2024

(54) SOLID HAND AND BODY LOTION PELLETS

(71) Applicants: Brynn Albrecht, Eagle Mountain, UT (US); Laurel Albrecht, Eagle Mountain, UT (US)

(72) Inventors: Brynn Albrecht, Eagle Mountain, UT (US); Laurel Albrecht, Eagle Mountain, UT (US)

(21) Appl. No.: 18/525,495

(22) Filed: Nov. 30, 2023

Related U.S. Application Data
(60) Provisional application No. 63/432,056, filed on Dec. 12, 2022.

(51) Int. Cl. A61K 8/02; A61K 8/67; A61K 8/73; A61K 8/92

(52) U.S. Cl. CPC A61K 8/0233; A61K 8/678; A61K 8/732; A61K 8/922; A61K 8/925

(57) ABSTRACT

A solidified lotion is provided. The lotion includes a mixture of moisturizers and solidifying agents and forms a solid at room temperature. The lotion is manufactured into solid shapes which each include a single use quantity of lotion. The individual solid shapes are coated with a starch such as arrowroot powder. These individual shapes of solid lotion are sufficiently durable to not deform or stick to each other; allowing a large quantity of the solid lotion shapes to be packaged together without individual wrapping or packaging. During use, the lotion shapes melt when rubbed onto a person's skin and allow for quick absorption into the skin.

### PRIORITY

The present application claims the benefit of U.S. Provisional Application Ser. No. 63/432,056, filed Dec. 12, 2022, which is herein incorporated by reference in its entirety.

### THE FIELD OF THE INVENTION

The present invention relates to hand and body lotions. In particular, examples of the present invention relate to a hand and body lotion which is provided in individual solid portions.

### INTRODUCTION

Hand and body lotions are commonly used to smooth and hydrate the user's skin. There is market demand for alternative forms of lotion which still function well to hydrate and condition the user's skin.

### BRIEF DESCRIPTION OF THE DRAWINGS

Non-limiting and non-exhaustive examples of the present invention are described with reference to the following figures, wherein like reference numerals refer to like parts throughout the various views unless otherwise specified.

FIG. 1 is a cross-sectional drawing of a lotion pellet.

FIG. 2 is a drawing which shows a number of lotion pellets in a container.

Corresponding reference characters indicate corresponding components throughout the several views of the drawings. Unless otherwise noted, the drawings have been drawn to scale. Skilled artisans will appreciate that elements in the figures are illustrated for simplicity and clarity. For example, the dimensions of some of the elements in the figures may be exaggerated relative to other elements to help improve understanding of various examples of the present invention. Also, common but well-understood elements that are useful or necessary in a commercially feasible embodiment are often not depicted in order to facilitate a less obstructed view of these various embodiments of the present invention.

It will be appreciated that the drawings are illustrative and not limiting of the scope of the invention which is defined by the appended claims. The examples shown each accomplish various different advantages. It is appreciated that it is not possible to clearly show each element or advantage in a single figure, and as such, multiple figures are presented to separately illustrate the various details of the examples in greater clarity. Similarly, not every example need accomplish all advantages of the present disclosure.

### DETAILED DESCRIPTION

In the following description, numerous specific details are set forth in order to provide a thorough understanding of the present invention. It will be apparent, however, to one having ordinary skill in the art that the specific detail need not be employed to practice the present invention. In other instances, well-known materials or methods have not been described in detail in order to avoid obscuring the present invention.

In the above disclosure, reference has been made to the accompanying drawings, which form a part hereof, and in which are shown by way of illustration specific implementations in which the disclosure may be practiced. It is understood that other implementations may be utilized and structural changes may be made without departing from the scope of the present disclosure. References in the specification to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, such feature, structure, or characteristic may be used in connection with other embodiments whether or not explicitly described. The particular features, structures or characteristics may be combined in any suitable combination and/or sub-combinations in one or more embodiments or examples. It is appreciated that the figures provided herewith are for explanation purposes to persons ordinarily skilled in the art.

As used herein, “adjacent” refers to near or close sufficient to achieve a desired effect. Although direct contact is common, adjacent can broadly allow for spaced apart features.

As used herein, the singular forms “a,” and, “the” include plural referents unless the context clearly dictates otherwise.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be such as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a number or numerical range endpoint by providing that a given value may be a significant figure above or a significant figure below the number or endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Dimensions, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range.

Many individuals use lotion to moisturize and condition skin. Liquid lotions are commonly used, but these can suffer from separation, drying at the nozzle, and can be inconvenient to carry or use at times. The present application discloses a solid, single use size lotion which may be used as a hand or body lotion. The lotion is solid at room temperature and is molded or otherwise formed into rounded or shaped pellets or beads. Typically, a person will take a bead of the lotion from a storage container holding many lotion beads and rub the lotion bead between their hands. While the lotion is solid at room temperature, contact with skin at body temperature melts the lotion and allows the lotion to absorb into the user's skin easily. The lotion is manufactured into single use pellets/beads which are provided in a larger container having many individual use pellets. The individual use lotion pellets are formulated so that they do not stick to each other in the container. This allows the individual lotion pellets to be easily dispensed and used without mess or hassle. The lotion pellet formulation allows the lotion pellets to be packaged together without requiring individual wrapping or using a tray or liner to separate individual lotion pellets. Each lotion pellet consists only of the lotion ingredients which are applied to the skin and the lotion pellets do not require a disposable plastic bead, pouch, or other individual container. The term pellet is used herein to refer to a shaped quantity of solid lotion. The lotion may be molded to a desired shape, etc.

The body lotion includes moisturizers, solids, solidifying agents, and an outer powder coating. The lotion pellets include mango butter, coconut oil, hemp seed oil, avocado oil, shea butter, and vitamin E as moisturizers. The lotion pellets include beeswax and cocoa butter solids and includes a starch powder solidifying agent. The starch powder may be arrowroot powder, tapioca powder, corn, potato, or rice starch, and is preferably arrowroot powder. The lotion pellets also may include a desired fragrance or color additive. The example lotion pellets may be formulated with mica colorants. The above ingredients are combined and form the solid lotion. In addition to the ingredients listed above, the lotion pellets include a coating of starch powder such as arrowroot powder around the outside of the lotion pellets.

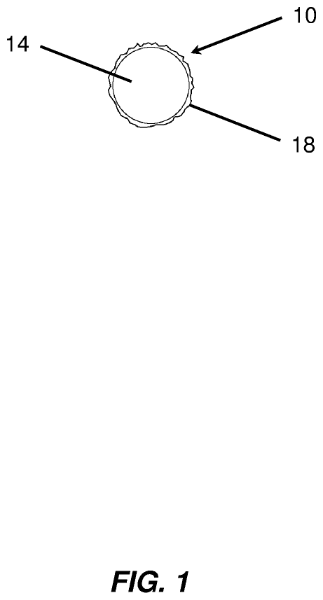

FIG. 1 shows a cross-sectional drawing of
an example solid lotion pellet 10. A round pellet of solid lotion 10 is shown, but other molded or formed shapes can also be used. The lotion pellet 10 includes a shaped portion of solid lotion 14 which includes moisturizers, moisturizing solids, and solidifying agents and may also include fragrance and colorants. The lotion body also includes a coating 18 of starch powder such as arrowroot powder. An example solid lotion pellet includes a solid lotion 14 that includes mango butter, coconut oil, hemp seed oil, avocado oil, shea butter, and vitamin E as moisturizers, includes beeswax and cocoa butter moisturizing solids, and includes a starch powder solidifying agent. The starch powder may be arrowroot powder, tapioca powder, corn, potato, or rice starch, and is preferably arrowroot powder. The solid lotion also may include a desired fragrance or color additive. The ingredients for the solid lotion 14 are typically heated and mixed together, placed into a mold to create portion sized amounts of lotion 14, and then cooled to solidify the mixture into solid lotion 14. The solid lotion pellets are then coated in a starch powder 18 which is preferably arrowroot powder.

The solid lotion 14 typically include between about 60 percent and about 75 percent moisturizers by weight, between about 20 percent and about 30 percent solids by weight, and between about 5 percent and about 15 percent starch powder by weight. Preferably, the starch powder is between about 5 percent and about 10 percent by weight. More particularly, the solid lotion 14 may include about 25 percent solids by weight and about 7 percent or about 8 percent starch powder solidifying agent by weight. Where the lotion includes fragrance and colorants, these are typically between about 5 percent and about 10 percent of the lotion by weight. With fragrance, the starch powder solidifying agent is about 7 percent of the solid lotion 14 by weight.

In one example formulation, the lotion pellets may include about 2600 grams of moisturizers, about 1000 grams of moisturizing solids, about 300 grams of starch powder, and about 300 grams of fragrance. The weight of the starch powder solidifying agent is between about 5 percent and about 15 percent of the combined weight of the moisturizers and moisturizing solids, is more particularly between about 5 percent and about 10 percent of the combined weight of the moisturizers and solids, and is preferably about 8 percent of the combined weight of the moisturizers and solids by weight. In one example formulation, the lotion pellets include approximately 1 part cocoa butter, 2 parts beeswax, and 1 part starch powder for approximately 8 parts moisturizers.

The beeswax and cocoa butter solids help to solidify the moisturizers to create a solid lotion. The arrowroot powder helps give structure and a desired consistency to the solidified lotion pellet. The arrowroot powder also helps provide a desired consistency to the lotion during application to a person's skin. The arrowroot powder helps the lotion spread onto the skin and helps the lotion absorb into the skin.

Lotion pellets are made by heating and mixing the moisturizers and solidifying agents together and then molding or otherwise forming the lotion into separate single use quantities of lotion. The lotion pellets may be molded into a desired shape and quantity, a desired quantity may be placed onto a surface to cool, etc. The solid lotion 14 is formulated to be solid at a room temperature which is about 25 degrees Celsius and to melt before body temperature of about 35 degrees Celsius. Once the solid lotion pellets are made, the exterior of the lotion pellets are coated in additional starch powder/arrowroot powder. The exterior coating of arrowroot powder stabilizes the surface of the solid lotion pellets and keeps the individual lotion pellets from sticking to each other inside of the packaging. The exterior coating of starch powder forms a significant part of the resulting lotion pellet. Single use lotion pellets 10 sized for application to the hands are often a sphere about 0.3 inches in diameter or a half sphere about 0.4 inches in diameter. A coating of starch powder 18 which is a few hundredths of an inch thick applied to a body of solid lotion 14 in these sizes is between about 5 percent and about 15 percent or more of the volume of the resulting lotion pellet 10, and is often about 10 percent of the volume of the resulting lotion pellet 10.

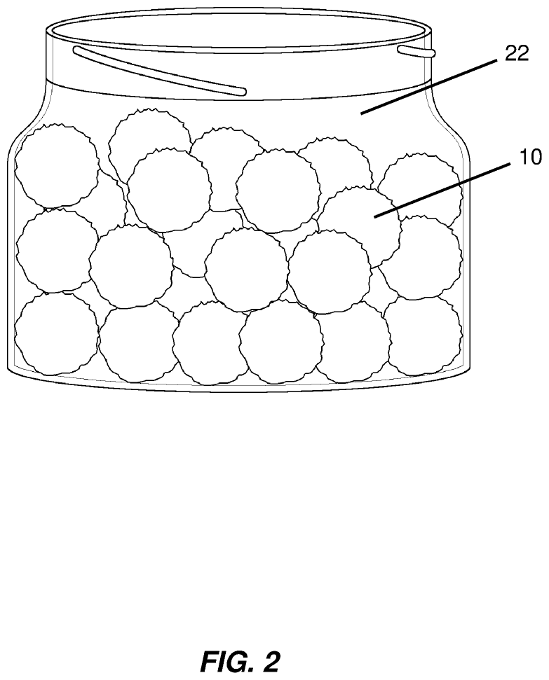

As shown in FIG. 2, the lotion pellets 10 may be packaged together with a larger quantity of lotion pellets 10 in a container 22. The lotion pellets are sufficiently hard to not deform while packaged together and do not stick to each other due to the starch powder coating 18 surrounding the solid lotion 14. Applicant has found that even after prolonged storage, the lotion pellets 10 remain separate and are substantially unattached to each other. A user may open the container, remove a single lotion pellet, and rub that lotion pellet onto their skin. The heat from their body and the friction of rubbing the lotion pellet on their skin easily melts the lotion pellet, allowing the moisturizers to soak into their skin. The starch powder coating 18 helps to provide an appealing hand feel to the user and also helps the lotion to melt and distribute itself around the user's skin. The starch powder which is mixed into the solid lotion 12 also helps provide a desirable feel to the lotion at is melts and is absorbed into the skin. The starch powder helps to prevent a greasy feel and promotes quick absorption into the skin.

The above description of illustrated examples of the present invention, including what is described in the Abstract, is not intended to be exhaustive or to be limiting to the precise forms disclosed. While specific examples of the invention are described herein for illustrative purposes, various equivalent modifications are possible without departing from the broader scope of the present claims. Indeed, it is appreciated that specific example materials, values, etc., are provided for explanation purposes and that other values may also be employed in other examples in accordance with the teachings of the present invention.

What is claimed is:

1. A solid lotion pellet comprising:
a solid lotion body comprising a mixture of:
a moisturizer;
a moisturizing solid; and
a starch powder solidifying agent;
wherein the moisturizer, moisturizing solid, and starch powder solidifying agent are mixed together and form a solid mixture at about 25 degrees Celsius and melt at about 35 degrees Celsius;
a coating of starch powder covering the exterior surface of the solid lotion body.

2. The solid lotion pellet of claim 1, wherein the moisturizer comprises at least one of the group consisting of mango butter, coconut oil, hemp seed oil, avocado oil, shea butter, and vitamin E.

3. The solid lotion pellet of claim 1, wherein the moisturizing solid comprises at least one of the group consisting of beeswax and cocoa butter.

4. The solid lotion pellet of claim 1, wherein the starch powder solidifying agent comprises at least one of the group consisting of arrowroot powder, tapioca powder, corn starch, potato starch, and rice starch.

5. The solid lotion pellet of claim 1, wherein the solid lotion body comprises:
between about 60 percent and about 75 percent moisturizer by weight;
between about 20 percent and about 30 percent moisturizing solid by weight; and
between about 5 percent and about 15 percent starch powder by weight.

6. The solid lotion pellet of claim 1, wherein the solid lotion body comprises between about 5 percent and about 10 percent starch powder by weight.

7. The solid lotion pellet of claim 5, wherein the solid lotion body comprises about 7 percent starch powder solidifying agent by weight.

8. The solid lotion pellet of claim 1, wherein the coating of starch powder comprises about 10 percent of the solid lotion pellet by volume.

9. The solid lotion pellet of claim 1, wherein the coating of starch powder comprises arrowroot powder.

10. The solid lotion pellet of claim 1, wherein the solid lotion body comprises about 1 part starch powder solidifying agent, about 2 parts beeswax, about 1 part cocoa butter, and about 8 parts moisturizer.

11. A solid lotion pellet comprising:
a solid lotion body comprising a mixture of:
a moisturizer;
a moisturizing solid; and
a starch powder solidifying agent, wherein the starch powder solidifying agent comprises between about 5 percent and about 15 percent of the solid lotion body by weight; and
wherein the moisturizer, moisturizing solid, and starch powder solidifying agent are mixed together and form a solid mixture at about 25 degrees Celsius and melt at about 35 degrees Celsius.

12. The solid lotion pellet of claim 11, wherein the moisturizer comprises at least one of the group consisting of mango butter, coconut oil, hemp seed oil, avocado oil, shea butter, and vitamin E;
wherein the moisturizing solid comprises at least one of the group consisting of beeswax and cocoa butter; and
wherein the starch powder solidifying agent comprises at least one of the group consisting of arrowroot powder, tapioca powder, corn starch, potato starch, and rice starch.

13. The solid lotion pellet of claim 11, wherein the solid lotion body comprises:
between about 60 percent and about 75 percent moisturizer by weight; and
between about 20 percent and about 30 percent moisturizing solid by weight.

14. The solid lotion pellet of claim 11, further comprising a coating of starch powder covering the exterior surface of the solid lotion body.

15. The solid lotion pellet of claim 14, wherein the coating of starch powder comprises about 10 percent of the solid lotion pellet by volume.

16. A solid lotion pellet comprising:
a solid lotion body comprising a mixture of:
a moisturizer;
a moisturizing solid; and
wherein the moisturizer and moisturizing solid are mixed together and form a solid mixture at about 25 degrees Celsius and melt at about 35 degrees Celsius;
a coating of starch powder covering the exterior surface of the solid lotion body.

17. The solid lotion pellet of claim 16, wherein the coating of starch powder comprises about 10 percent of the solid lotion pellet by volume.

18. The solid lotion pellet of claim 16, wherein the solid lotion body further comprises a starch powder solidifying agent mixed with the moisturizer and the moisturizing solid.

19. The solid lotion pellet of claim 18, wherein the solid lotion body comprises between about 5 percent and about 10 percent starch powder by weight.

20. The solid lotion pellet of claim 18, wherein the starch powder solidifying agent comprises at least one of the group consisting of arrowroot powder, tapioca powder, corn starch, potato starch, and rice starch.
